SEQUENCE LISTING (4 sequences)

SEQ ID NO: 1 - DNA, 20 bp, Artificial
  Synthetic oligonucleotide
tggaactggctgaactgaca

SEQ ID NO: 2 - DNA, 20 bp, Artificial
  Synthetic oligonucleotide
agttgatggcttccttgtgg

SEQ ID NO: 3 - DNA, 17 bp, Artificial
  Synthetic oliognucleotide
aggtccgctcgctgagg

SEQ ID NO: 4 - DNA, 19 bp, Artificial
  Synthetic oligonucleotide
gttccagcgtgacgttttg

(19) United States
(12) Patent Application Publication
(10) Pub. No.: US 2007/0209084 A1
(43) Pub. Date: Sep. 6, 2007

(54) Adrenergic Receptor SNP for Improved Milking Characteristics

(76) Inventors: Robert J. Collier, Tuczon, AZ (US); Michael M. Lohuis, Des Peres, MO (US); Michael D. Grosz, Ellisville, MD (US)

Correspondence Address: QUARLES & BRADY LLP, 33 E. MAIN ST, SUITE 900, P.O BOX 2113, MADISON, WI, 53701-2113, US

(21) Appl. No.: 10/572,989

(22) PCT Filed: Sep. 21, 2004
(86) PCT No.: PCT/US04/30774

§ 371 Date: Jan. 25, 2007

(51) Int. Cl. A01K 67/027; C12Q 1/68

(52) U.S. Cl. 800/15; 435/6

(57) ABSTRACT

Disclosed herein is a method for screening for the allele associated with a desired SCS phenotype, which comprises: obtaining a DNA sample from a bull to be tested for the desired SCS phenotype; and detecting the presence of an adenine at position 11 in a gene encoding a bovine beta2-adrenoreceptor. Also disclosed is a milling attribute PCR-RFLP kit containing a pair of primers which flank the 11^{th} nucleotide position of the bovine beta2-adrenoreceptor gene, and a restriction enzyme specific for the CCCGGG site, which can be SmaI.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

The present invention relates, in general, to the field of molecular biology, human and bovine genetics, and desirable milking characteristics. In particular this invention provides a method for using genetics to predict the milling characteristics of cows.

Various publications or patent are referred to throughout this application to describe the state of the art to which the invention pertains. Each of this publications or patents is incorporated by reference herein. Complete citations of scientific publications are set forth in the text or at the end of the specification.

Domestic animals are bred for improvement to the useful attributes of the animals. For cows, one very useful attribute is their milking characteristics, both volume of milk and rate of milking. Many dairy cows are artificially inseminated using semen from sires purchased from companies engaged in selling quality improvement genetics for milking cows. However, while the overall statistical average of the milking characteristics of the daughters of a particular sire can be measured, it is yet not possible to predict with any degree of success the milking characteristics of a particular progeny created by breeding a cow with a particular bull. Better understanding of the genetics of milking traits will be needed in order to more accurately predict the milking characteristics of a particular daughter.

It is known that some milking characteristics are linked to other traits. A relationships between adrenoceptor concentrations and milkability in primiparous cows have been elucidated by Roets, et al.(“Relationship between milkability and adrenoceptor concentrations in teat tissue in primparous cows”, J Dairy Sci 69(12):2131-3131, 1986). Later, Roets et al. (“Relationship between numbers of alpha2- and beta2-adrenoceptors in teat tissue and blood cells and milkability of primiparous cows”, J Dairy Sci 72(12):3304-33-13, 1989) reported on the numbers of alpha2- and beta2-adrenoceptors in teat tissue and blood cells and milkability in primiparous cows. Subsequently, Roets et al.(“Relationship between numbers of alpha2- and beta2-adrenoceptors on blood cells of bulls and milkability of their daughters”, J Dairy Sci, 62 567-575, 1995) analyzed the numbers of alpha2- and beta2-adrenoceptors on the blood cells of bulls and the milkability of their daughters. In addition, Blum et al.(“Catecholamines, oxytocen and milk removal in dairy cows”, J Dairy Res, 56(2):167-177, 1989) reported that treatment with phentolamine (an alpha-adrenergic blocker) and isoproterenol (a beta-adrenoceptor agonist) each facilitated milk removal. Brown et al. (“Relationship of milking rate to somatic cell count”, J Dairy Sci, 69(3):850-854, 1986) raised the possibility that milking speed was correlated with both somatic cell score (SCS) and mastitis. Many studies have established correlations between mastitis and SCS (see, for example, MacMillan et al., “Associations between dry cow therapy, clinical mastitis, and somatic cell count score with milk and fat production in ten New Zealand dairy herds”, J Dairy Sci, 66(2):259-265, 1983; Reneau, “Effective use of dairy herd improvement somatic cell counts in mastitis control”, J Dairy Sci, 69(6):1708-1720, 1986; and McClelland, “A comparison of objective and subject measures of milking speed on Canadian Holstein-Friesians”, University of Guelph, 1983). Further, a positive correlation between milking speed and SCS was confirmed by Boettcher et al. (“Development of an udder health index for sire selection based on somatic cell score, udder conformation, and milking speed”, J Dairy Sci, 81(4):1157-1168, 1998).

The DNA sequence in and around the bovine beta2-adrenergic receptor (ADRB2) gene was in public genome databases (accession numbers Z86037; Einspanier et al., “Expression of the beta2 adrenergic receptor in the cattle”, direct submission to Genbank, accession No. Z86037, 1997 and AF331034; Schimpf et al., “Genetic mapping of the ADRB2 gene on cattle chromosome 7”, Anim. Genet. 32(6):390, 2001). These sequences included the ADRB2 coding region (1257 bases inclusive from the ATG start to the TAA stop codon), as well as the 223 bases upstream from the ATG start codon and the 550 bases downstream from the TAA stop codon.

What is needed is a way of determining which bulls will produce cows with improved milkability and a method of improving the results of a breeding program for milkability.

### BRIEF SUMMARY OF THE INVENTION

Disclosed herein is a method for breeding cows for improved milking characteristics including the step of screening the genotype of the parents of the cow for the allele associated with a desired SCS phenotype. The method has the steps of obtaining a DNA sample from a bull to be tested for the desired SCS phenotype; and detecting the presence of an adenine at position 11 in a gene encoding a bovine beta2-adrenoreceptor. The method can be performed by direct sequencing, primer extension, restriction length fragment polymorphism, and allele-specific hybridization.

In another embodiment, a method of identifies a bull whose daughter cows have a short milking duration This method has the steps of obtaining a sample of DNA from a bull; combining the DNA with a pair of PCR probes comprising SEQ IDs 1 and 2 or SEQ IDS 3 and 4; incubating the DNA under conditions permitting the DNA bounded by the PCR probes to produce DNA amplicons; isolating the DNA amplicons; combining the DNA amplicons with a restriction enzyme specific for CCCGGG for a sufficient time to produce a mixture of DNA fragments from the amplicons comprising CCCGGG; applying the DNA fragment mixture to a gel and permitting migration of the mixture components for a time sufficient for them to separate; and observing the sizes of DNA on the gel, with the largest fragments being correlated with the A genotype and with better SCS phenotype, and the smaller fragments being associated with the C genotype and less desirable SCS phenotype.

In another embodiment a milking attribute PCR-RFLP kit contains a pair of primers which flank the 11^{th} nucleotide position of the bovine beta2-adrenoreceptor gene, and a restriction enzyme specific for the CCCGGG site. The restriction enzyme can be SmaI. The primer pairs are selected from pair 1 (SEQ ID Nos 1 and 2) or from pair 2 (SEQ ID Nos 3 and 4).

Other objects, features and advantage of the present invention will become apparent to one of skill in the art after review of the specification and claims.

### BRIEF DESCRIPTION OF THE SEVERAL VIEWS OF THE DRAWINGS

Not applicable.

### DETAILED DESCRIPTION OF THE INVENTION

It is disclosed here that genetic differences in the allele for the beta2-adrenoceptor (ADR2) gene are associated with the milking characteristics of milking cows. This makes possible the use of genetic tools and analysis in breeding of cows to start to predict with accuracy the milking characteristics of daughters of specific bulls bred to specific maternal cows.

We began the work described here by proposing that DNA sequence polymorphisms in and around bovine beta2-adrenoceptor (ADRB2) may cause or be associated with genes that cause the observed associations. First, we subcloned and sequenced bovine ADRB2, extending the region of known sequence beyond what was previously publicly available. Sequence obtained through this work, not previously in the public domain, includes 876 additional bases upstream from that described above. These bases are a total of 1099 that are 5′ to the ATG start codon. An additional 695 bases downstream from the previously reported bases also were sequenced. These bases include a total of 1245 bases 3′ to the TAA stop codon.

With this information in hand, we were then able to design oligonucleotide primers to be used to amplify and perform locus-specific re-sequencing of genomic DNA spanning regions of the bovine ADRB2 gene. These primers were used to amplify genomic DNA from 24 Holstein and 12 Brown Swiss dairy cows. After comparative genomic sequence was obtained, we analyzed the sequence using polyPhred software (Washington University, St. Louis, Mo.) and identified, among others, a Single Nucleotide Polymorphism (SNP) sequence at position 11, inclusive of the start ATG (hereafter referred to as “A11C”). The published sequence indicates the presence of a cytosine (C) nucleotide. We discovered a variant allele with an adenine (A) nucleotide at that position. Substitution of A for C at the nucleotide position 11 altered the amino acid sequence of ADRB2 protein by replacing a proline with a histidine at amino acid position 4 of the protein.

Subsequently we designed and validated a polymerase chain reaction-restriction fragment length polymorphism (PCR-RFLP) assay able to detect the presence of A and/or C nucleotide(s) at the A11C locus. This assay is based on the presence or absence of the smaI restriction enzyme recognition site (CCCGGG) in DNA fragments which have been amplified by one of two primer pairs. The A allele at the A11C locus changes this sequence to CACGGG, which is not recognized, therefore is not digested by SmaI and consequently is identified as a heavier nucleotide in the PCR-RFLP test.

This polymorphism was found to be associated with milking characteristics. In particular, the A allele at the A11C locus is associated with higher somatic cell score (SMS) and therefore with a faster milking speed. This makes is possible to breed dairy cattle to increase the proportion of the herds having the A allele at the A11C locus, to improve the overall milking speed of the herd. The A allele is effective in both the heterozygous and homozygous conditions to improve the performance of the animal.

“Tolymorphism” refers generally to the ability of an organism or gene to occur in two or more different forms. In particular for purposes of the present invention, “polymorphism” refers to two or more different forms of the same gene.

“Single Nucleotide Polymorphism” or “SNP” refers to a polymorphism that results from a difference in a single nucleotide. [000211 A “Restriction Enzyme” refers to an endonuclease which bins to double stranded DNA at a specific nucleotide sequence and then, if both strands of the DNA lack the appropriate modification at that sequence (including but not limited to methylation), cleaves the DNA either at the recognition sequence or at another site in the DNA molecule. Restriction enzymes are denoted by three-letter abbreviations followed by a strain designation and/or a Roman numeral distinguishing different enzymes from the same species or strain. Recognition sequences are written 5′ to 3′ for one strand only. Examples of restriction enzymes include SmaI, BamHi, BclI, EcoRI, HindIII, and XbaI.

The term “Allele” refers generally to any of one or more alternative forms of a given gene or DNA segment; both or all alleles of a given gene are concerned with the same trait or characteristic, but the product or function coded for by a particular allele differs qualitatively and/or quantitatively from that coded for by other alleles of that gene. In a diploid cell or organism the members of an allelic pair (i.e., the two alleles of a given gene) occupy corresponding positions (loci) on a pair of homologous chromosomes; if these alleles are genetically identical the cell or organism is said to be homozygous. If the alleles are genetically different, the cell or organism is said to be heterozygous with respect to the particular gene.

The polynucleotides of the present invention may be prepared by two general methods: (1) They may be synthesized from appropriate nucleotide triphosphates, or (2) they may be isolated from biological sources. Both methods utilize protocols well known in the art. The availability of nucleotide sequence information enables preparation of an isolated nucleic acid molecule of the invention by oligonucleotide synthesis. Synthetic oligonucleotides may be prepared by the phosphoramidite method employed in the Applied Biosystems 37A DNA Synthesizer or similar devices. The resultant construct may be purified according to methods known in the art, such as high performance liquid chromatography (HPLC). Complementary segments thus produced may be annealed such that each segment possesses appropriate cohesive termini for attachment of an adjacent segment. Adjacent segments may be ligated by annealing cohesive termini in the presence of DNA ligase to construct an entire long double-stranded molecule. A synthetic DNA molecule so constructed may then be cloned and amplified in an appropriate vector.

The invention will be better understood upon consideration of the following non-limiting Examples. Unless otherwise specified, general cloning procedures, such as those set forth in Sambrook et al., MOLECULAR CLONING, Cold Spring Harbor Laboratory (1989) or Ausubel et al. CURRENT PROTOCOLS IN MOLECULAR BIOLOGY, John Wiley & Sons (2001), are used.

### EXAMPLE 1

### Assessment of mRNA Levels in Leukocytes of Cows with Fast vs. Slow-Milking Rates

Eight animals were selected by the duration of their milking and grouped as either slow or fast. For initial determinations, slow-milking animals were numbered 90, 264, 279 and 321. Fast-milking animals were numbered 272, 273, 281 and 289. Their blood (about 15 mL) was collected in EDTA-filled Vacutainers (BD, Franklin Lakes, N.J.). Known sequences from NCBI along with the program “Oligo” were used to design primer sets specific for GAPDH, alpha2-, beta 1-, beta2-adrenoreceptors, and beta-arrestin.

From the blood samples, RNA was isolated using the RNeasy RNA isolation protocol (Qiagen, Valencia, Calif.). One volume of whole blood was mixed with 5 volumes of erythrocyte lysis buffer. The mixture was incubated on ice, and during the incubation mixed by vortexing briefly twice. The mixture was next centrifuged at 400 g for 10 min at 4° C. The supernatant was discarded and the leukocyte pellet saved. The leukocyte pellet was added to more Buffer EL and vortexed to resuspend the leukocytes. The mixture was again centrifuged, after which the supernatant was discarded. The leukocyte pellet was loosened by flicking the tube. Buffer RLT (Qiagen)(with 10 μl β-mercaptoethanol added per 1 ml buffer) was added to the leukocytes; for a starting blood volume of 4.0 ml or less, 2.0 ml was added; and for a greater blood volume, 4.0 ml was added. Next the mixture was homogenized until the sample was homogeneous. An equal volume of 70% alcohol was added to the homogenized lysate and vigorously shaken. Finally, the sample, including any precipitate that may have formed, was applied to an RNeasy midi spin column placed in a 15-ml centrifuge tube and centrifuged for 3000 to 5000 g for 5 min. For samples in excess of 4.0 ml, the extra sample was applied successively to the same column and the flow-through discarded. After the flow-through had been discarded, 4.0 ml of Buffer RW1 was added to the RNeasy column and centrifuged at 3000-5000 for 5 min to wash the column. The flow-through was discarded. Next 2.5 ml of Buffer RPE (diluted with 4 volumes of ethanol (96-100%) was added to the column which was again centrifuged at 3000-5000 for 2 min. Another 2.5 ml Buffer RPE was added to the column which was centrifuged at 3000-5000 for 5 min to dry the spin-column membrane. RNA was eluted from the column adding 150 μl or 250 μl RNase-free water to the column, which was left to stand for 1 min and then centrifuged at 3000-5000 g for 3 min. A second volume of RNase-free water was added to the column and spun down. The RNA was then salt-precipitated according to the manufacturer's protocol.

Next RNA was treated with DNA-free DNase to remove trace levels of DNA, according to the manufacturer's protocol (Ambion, Austin, Tex.). To the RNA sample purified as described above, were added 0.1 volume of 10× DNase I Buffer and 1 μl of DNase I (2 units) to the RNA, which solution was mixed gently and incubated at 37° C. for 20-30 min. Ambion DNase Inactivation reagent was resuspended by flicking or vortexing. From that tube, the greater of 0.1 volume or 5 μl was added to the sample and mixed well. The tube was incubated for 2 min at room temperature. The tube was next centrifuged at 10,000 g for about 1 min to pellet the DNase inactivation reagent.

Spectrophotometry was then used to determine RNA concentration, and 1 μg RNA was loaded on a 1% agarose gel using ethidium bromide to ensure quality.

Following the manufacturer's protocol, cDNA templates were made from the RNA using Omniscript (Qiagen) for Real Time PCR analysis. The Omniscript 10× Buffer RT, dNTP mix and RNase-free water were first thawed and mixed by vortexing. Qiagen RNase inhibitor was diluted to a final concentration of 10 units/μl in 1× Buffer RT and mixed by vortexing briefly. Master mix (2.0 μl of 10× Buffer RT, 2.0 μl dNTP mix, 2.0 μl oligo dT primer (10 uM), 0.5 μl Rnase inhibitor (10 units/μl), 1.0 μl Omniscript reverse transcriptase and double distilled water) was prepared on ice. Master solution was distributed to the various tubes, and template RNA (0.6 to 0.68 μl/tube) was added and mixed. The resulting solutions were incubated at 37° C. for 60 min.

All samples were run by LightCycler (Idaho Technology, Inc., Salt Lake City, Utah) protocol using the SYBR-green kit from Roche Molecular Biochemicals, except for beta2-adrenoreceptor, for which the SYBR-green kit (Qiagen) was used. Samples were run in duplicate, and all were normalized using GAPDH. Cycle thresholds (determined by the second derivative growth curve) of samples were then compared between the groups of slow and fast milkers. To determine if there were any significant differences between slow and fast milkers, the SAS statistical package was used to perform a Pearson correlation coefficient. The results suggested that duration of milking and rate of milk removal are associated with the amount of beta2-adrenoreceptor mRNA levels in blood, and it was decided to more thoroughly characterize the beta2-adrenoreceptor gene in cattle.

It was discovered that there was a polymorphism of the beta2-adrenoreceptor at the 11^{th} nucleotide (A replaced C in some instances, resulting in a substitution of histidine for proline at the fourth amino acid).

### EXAMPLE 2

### Associations Between A11C Genotype and SCS and/or Milking Speed in Dairy Cows

Bovine DNAs are available from the Cooperative Dairy DNA Repository (CDDR) population (Gene Evaluation and Mapping Laboratory, Bldg. 200 Rm 2A, ARS-USDA, BARC-East Beltsville, Md. 20705). SCS phenotypes have been obtained for all CDDR animals. For a subset of the CDDR animals, there also are data on the milking speed (MS). First, CDDR animals with MS data were genotyped, along with a subset of the remainder of the CDDR animals representing “high” and “low” Somatic Cell Score (SCS) phenotypic classes, with the A11C assay. DNA samples were obtained and assayed. Data sets were distributed and analyzed in duplicate. The analysis revealed a correlation between A11C and SCS.

Six hundred sixty three animals from the CDDR were genotyped for the A11C locus using the either of the following pairs of primers:

| | |
|---|---|
| TGGAACTGGCTGAACTGACA | (SEQ ID NO 1) |
| AGTTGATGGCTTCCTTGTGG | (SEQ ID NO 2) |
| AGGTCCGCTCGCTGAGG | (SEQ ID NO 3) |
| GTTCCAGCGTGACGTTTTG | (SEQ ID NO 4) |

Table 1 shows a disproportionate distribution of A11C genotypes into “High” and “Low” SCS phenotypic categories.

TABLE 1
A11C Genotypes and SCS distribution of 663 CDDR bulls.

| | AA | AC | CC | Total |
|---|---|---|---|---|
| High # | 9 | 88 | 248 | 345 |
| Low # | 5 | 55 | 258 | 318 |
| Total | 14 | 143 | 506 | 663 |
| Prop. High | 0.643 | 0.615 | 0.490 | 0.520 |
| Prop. Low | 0.357 | 0.385 | 0.510 | 0.480 |

In addition, Table 2 contains average estimated transmitting abilities (ETA) for SCS and frequencies by A11C genotype. Results from statistical analyses of the data indicate that genotypes AA, AC and CC are significantly different from the mean SCS (p<0.025) and that genotypes AA and AC are significantly higher than the mean SCS (p<0.01). Furthermore, animals with the CC genotype have significantly different (i.e., lower) SCS phenotypes than animals with AA and AC genotypes (p<0.05).

TABLE 2
Average SCS ETA and frequency
by A11C genotype among 663 CDDR bulls.

| | AA | AC | CC | Total |
|---|---|---|---|---|
| SCS ETA | 314.5 | 312.8 | 307.0 | 0.520 |
| Genotype Freq. | 0.021 | 0.216 | 0.763 | |

Table 3 shows the distribution of milking speed (MS) classifications according to genotype, and Table 4 illustrates the average ETA for MS by genotype. Because of the small number of AA animals, there is not a significant association between MS and A11C genotype at the 5% level. However, the trend in Table 3 clearly shows that the CC genotype has a lower proportion of bulls transmitting fast MS.

TABLE 3
A11C Genotypes and Milking Speed distribution of 663 CDDR bulls.

| | AA | AC | CC | Total |
|---|---|---|---|---|
| Fast # | 13 | 46 | 108 | 167 |
| Slow # | 7 | 36 | 120 | 163 |
| Total | 20 | 82 | 228 | 330 |
| Prop. Fast | 0.650 | 0.561 | 0.474 | 0.506 |
| Prop. Slow | 0.350 | 0.439 | 0.526 | 0.494 |

In Table 4, the AA genotype tends to have a higher average ETA for MS.

TABLE 4
Average ETA for milking speed and frequency
by A11C genotype among 663 CDDR bulls.

| | AA | AC | CC |
|---|---|---|---|
| MS ETA | 70 | 68.81707 | 64.02609 |
| Genotype Freq. | 0.061 | 0.248 | 0.691 |

From these tables it appears that the AA and AC genotypes (that are significantly associated SCS) are also associated with higher milking speed. This is in agreement with studies (McClelland, “A comparison of objective and subject measures of milking speed on Canadian Holstein-Friesians”, University of Guelph, 1983, and Boettcher et al., “Development of an udder health index for sire selection based on somatic cell score, udder conformation, and milking speed”, J Dairy Sci 81(4):1157-1168, 1998) that have shown that higher milking speed is genetically correlated with higher SCS and mastitis incidence. Since the bulls that had MS ETAs represented only a subset (330) of the total group (663), it is likely that with a larger sample size, significant association could be shown between MS and the A11C locus.

This discovery enables for the first time the testing and selection of animals for breeding and/or alternative management practices based on the results of the inventive assay. The assay results correlate with the SCS and enable the choice of animals with improved MS and mastitis resistance. In addition to using these discoveries to select animals for phenotypes related to SCS, these discoveries can also be used to select animals in an attempt to effect population changes in MS and mastitis resistance. Applying the assay will enable the selection of animals with SCS which will ultimately improve the herd averages for MS and mastitis resistance.

This invention also includes a kit containing reagents that can be used to identify allelic composition at the loci described herein.

The present invention is not limited to the embodiments described and exemplified above, but is capable of variation and modification within the scope of the appended claims.

What is claimed is:

1. A method for breeding cows for desired milking characteristics comprising the steps of:
a. identifying the allele of the bovine beta2-adrenergic receptor gene in at least one potential parent animals; and
b. breeding male and female animals to create a daughter having at least one allele of the beta2-adregenic receptor gene associated with improved milking characteristics.

2. The method of claim 1 wherein the method for identifying the allele includes isolating DNA from the parent and screening with a method selected from the group consisting of direct sequencing, primer extension, restriction length fragment polymorphism, and allele-specific hybridization.

3. The method of claim 1 whereby the screening method is intended to identify A11C alleles.

4. A method of identifying a bull whose daughter cows will have a faster milking time, the method comprising the steps of
a. obtaining a sample of DNA from a bull;
b. combining the DNA with a pair of PCR probes comprising SEQ IDs 1 and 2 or SEQ IDS 3 and 4;
c. incubating the DNA under conditions permitting the DNA bounded by the PCR probes to produce DNA amplicons;
d. isolating the DNA amplicons;
e. combining the DNA amplicons with a restriction enzyme specific for CCCGGG for a sufficient time to produce a mixture of DNA fragments from the amplicons comprising CCCGGG;
f. applying the DNA fragment mixture to a gel and permitting migration of the mixture components for a time sufficient for them to separate; and
g. observing the sizes of DNA on the gel, with the largest fragments being correlated with the A genotype and with better SCS phenotype, and the smaller fragments being associated with the C genotype and less desirable SCS phenotype.

5. A milking attribute PCR-RFLP kit, which comprises:
a pair of primers which flank the 11^{th} nucleotide position of the bovine beta2-adrenoreceptor gene, and a restriction enzyme specific for the CCCGGG site.

6. The kit of claim 5 wherein the restriction enzyme is SmaI.

7. The kit of claim 5 wherein the primer pairs are selected from pair 1 (SEQ ID Nos 1 and 2) or from pair 2 (SEQ ID Nos 3 and 4).
